(19) United States
(12) Patent Application Publication
(10) Pub. No.: US 2010/0003204 A1
(43) Pub. Date: Jan. 7, 2010

(54) Nanoparticle hybrid sunscreens

(75) Inventors: Douglas Anson Loy, Tucson, AZ (US); Kenneth J. Shea, Irvine, CA (US); Dylan J. Boday, Tucson, AZ (US); Jason T. Wertz, Tucson, AZ (US)

Correspondence Address: ALBERTO A. LEON, ESQUIRE, P.O. BOX 30684, ALBUQUERQUE, NM, 87190, US

(73) Assignee: Energy Materials Corporation, Livermore, CA (US)

(21) Appl. No.: 12/459,541

(22) Filed: Jul. 2, 2009

Related U.S. Application Data
(60) Provisional application No. 61/133,875, filed on Jul. 2, 2008.

(51) Int. Cl. A61K 8/02; C01B 33/12

(52) U.S. Cl. 424/59; 423/335

(57) ABSTRACT

The invention discloses and claims a new class of chemical compositions with ideal sunscreen properties. The chemical composition comprises spherical particles between 200 nm and 10 microns in diameter prepared by an emulsion polymerization of tetraalkoxysilanes or organotrialkoxysilanes or organobridged trialkoxysilanes with a dye monomer bearing two or more alkoxysilyl groups attached to the bridging chromophore. The resulting spheres absorb ultraviolet light. The dye can be any organic chromophore capable of receiving multiple trialkoxysilyl groups resulting in multipoint covalent attachment that precludes leaching of the dye from the spheres. The formic acid-toluene-monomer emulsion polymerization allows for large-scale (>100 gram) synthesis of monodisperse particles under acidic, non-aqueous conditions without surfactants. The particles less than 1 micron in diameter are smooth as talc to the touch and will provide a smooth formulation for sunscreen creams or lotions.

### CROSS-REFERENCE TO RELATED APPLICATION

I hereby claim the benefit under Title 35, United States Code Section 119(e) of any United States Provisional Application(s) listed below:

### Application Number: 61/133,875

### Filing Date: Jul. 2, 2008

### TECHNICAL FIELD OF THE INVENTION

The present invention relates generally to nanoparticles and to sunscreen preparations; more particularly to nanoparticle sunscreen preparations; and even more particularly to a new family of sunscreen chemical compositions prepared using a surfactant-free, anhydrous, formic acid-toluene-alkoxysilane emulsion polymerization-based method.

### BACKGROUND OF THE INVENTION

The following description of the art related to the present invention refers to a number of publications and references. Discussion of such publications herein is given to provide a more complete background of the scientific principles related to the present invention and it is not to be construed as an admission that such publications are necessarily prior art for patentability determination purposes.

In modem times, sunscreens have become an integral part of many people's overall healthcare and disease prevention strategy. Sunscreens are known to reduce the harm caused by ultraviolet radiation during human skin's exposure to the sun. The first known commercial sunscreens physically blocked ultraviolet radiation and were petroleum based. The effectiveness of many modem sunscreen products is usually based on an organic chemical compound that absorbs in the ultraviolet spectrum and either fluoresces in non-harmful visible wavelengths or converts the energy absorbed into heat. Usually, a sunscreen preparation comprises such an organic compound(s) dissolved in lotion formulations (between 5 and 15 weight percentage), and are applied directly to the skin.

Specifically, the principal ingredients in modem sunscreens are usually aromatic molecules conjugated with carbonyl groups. That general structure allows the molecule to absorb high-energy ultraviolet rays and release the energy as lower-energy rays, thereby preventing the skin-damaging ultraviolet rays from reaching the skin. Some sunscreens also include enzymes like photolyase, which repair UV-damaged DNA.

Concerns about the health effects of the organic compounds in sunscreens have limited the number of products, and have slowed the approval of new compounds. The organic compounds in sunscreens could potentially have chronic effects including mutagenicity, steroid mimicry or toxicity. Micron or larger inorganic alternatives, such as titanium dioxide and zinc oxide, block sun rays through reflection, absorption of ultraviolet and Mie scattering, causing a white appearance. Those materials, however, are usually gritty, opaque and white. It is desirable to formulate grit and color-free, transparent sunscreens.

Nanoparticles (<100 mn) of titanium dioxide and zinc dioxide have recently been included in grit-free, non-colored, transparent sunscreen formulations. Although titanium dioxide absorbs ultraviolet light, it also catalyzes the formation of hydroxide radicals, which could present some obvious health concerns. The literature refers to health concerns regarding the use of nanoparticles, including their penetration of the skin and toxic properties in vivo. [Nohynek, Gerhard J.; Lademann, Jurgen; Ribaud, Christele; Roberts, Michael S. Grey goo on the skin? Nanotechnology cosmetic and sunscreen safety. Critical Reviews in Toxicology (2007), 37(3), 251-277] Those concerns may limit or prevent widespread acceptance of nanomaterials for use in topical formulations. A logical and rather simple way to address the health concerns involving the use of nanoparticles in sunscreen compositions is to find a way to prevent their penetration into the user's body. Several techniques have proven effective in encapsulating or binding sunscreen agents. The net effect of those techniques is to reduce those agents' reactivity and ability to enter the human body through the skin. Among the encapsulated sunscreens, publications have disclosed sunscreens physically encapsulated in silica shells, zeolite particles, lipids and other biomaterial particles or polymer shells. [(a) Lapidot, Noa; Gans, Orit; Biagini, Fabio; Sosonkin, Ludmila; Rottman, Claudio. Advanced sunscreens: UV absorbers encapsulated in sol-gel glass microcapsules. Journal of Sol-Gel Science and Technology (2003), 26(1/2/3), 67-72. (b) Chretien, Michelle N.; Migahed, Lamiaa; Scaiano, J. C. Protecting the protectors: reducing the biological toxicity of UV sunscreens by zeolite encapsulation. Photochemistry and Photobiology (2006), 82(6), 1606-1611. (c) Xia, Q.; Saupe, A.; Muller, R. H.; Souto, E. B. Nanostructures lipid carriers as novel carrier for sunscreen formulations. International Journal of Cosmetic Science (2007), 29(6), 473-482. (d) Lee, Wilson A.; Pernodet, Nadine; Li, Bingquan; Lin, Chien H.; Hatchwell, Eli; Rafailovich, Miriam H. Multicomponent polymer coating to block photocatalytic activity of TiO2nanoparticles. Chemical Communications (Cambridge, United Kingdom) (2007), (45), 4815-4817. (e) Gogna, Deepak; Jain, Sunil K.; Yadav, Awesh K.; Agrawal, G. P. Microsphere based improved sunscreen formulation of ethylhexyl methoxycinnamate. Current Drug Delivery (2007), 4(2), 153-159. (f) Ukaji, Emi; Harigae, Kazuhiko; Suzuki, Noboru; Furusawa, Takeshi; Sato, Masahide. The silica coating of fine TiO2 particles by sol-gel process and its effects on the photocatalytic activity and UV-shielding ability. Material Technology (Tokyo, Japan) (2006), 24(5), 275-281].

In each of the examples found in the literature, the sunscreen's organic composition can leak from the carrier leading to the same potential health problems cited above. [Benson, Heather A. E. Sunscreens: efficacy skin penetration and toxicological aspects. Dermatologic, Cosmeceutic, and Cosmetic Development (2008), 419-435.]. Encapsulation of titanium dioxide nanoparticles with silica shells adds a second step to the formation of the sunscreen making the material more costly to produce.

Dyes have been incorporated into silica nanoparticles by a number of methods. [(a) Shibata, S.; Taniguchi, T.; Yano, T.; Yamane, M. Formation of water-soluble dye-doped silica particles. Journal of Sol-Gel Science and Technology (1997), 10(3), 263-268. (b) Zhao, Xiaojun; Bagwe, Rahul P.; Tan, Weihong. Development of organic-dye-doped silica nanoparticles in a reverse microemulsion. Advanced Materials (Weinheim, Germany) (2004), 16(2), 173-176. (c) Ow, Hooisweng; Larson, Daniel R.; Srivastava, Mamta; Baird, Barbara A.; Webb, Watt W.; Wiesner, Ulrich. Bright and Stable Core-Shell Fluorescent Silica Nanoparticles. Nano Letters (2005), 5(1), 113-117. (d) Van Blaaderen, A.; Vrij, A. Synthesis and characterization of colloidal dispersions of fluorescent, monodisperse silica spheres. Langmuir (1992), 8(12), 2921-31. (e) Dewar, Patricia J.; MacGillivray, Tanya F.; Crispo, Sabrina M.; Smith-Palmer, Truis. Interactions of Pyrene-Labeled Silica Particles. Journal of Colloid and Interface Science (2000), 228(2), 253-258. (f) Jungmann, Nadja; Schmidt, Manfred; Ebenhoch, Jochen; Weis, Johann; Maskos, Michael. Dye loading of amphiphilic poly(organosiloxane) nanoparticles. Angewandte Chemie, International Edition (2003), 42(15), 1714-1717.] Those methods include physical encapsulation, which is prone to leaching. It also includes core shell forms that reduce but cannot eliminate leaching. Researchers have also utilized covalent incorporation of dyes, generally through surface modification of the particles or copolymerization with dye-modified trialkoxysilanes. In all of those cases, the particles are made either by Stober syntheses or through emulsion polymerizations. The present invention discloses and claims and novel and useful method, as none of the methods surveyed in the literature used formic acid to construct fluorescent or UV absorbing particles.

### SUMMARY OF THE INVENTION

Objects, advantages and novel features, and further scope of applicability of the present invention will be set forth in part in the detailed description to follow, taken in conjunction with the accompanying drawing. The objects, advantages and novel features, and further scope of applicability of the present invention will become apparent to those skilled in the art upon examination of the following, or may be learned by practice of the invention. The objects and advantages of the invention may be realized and attained by means of the instrumentalities and combinations particularly pointed out in the appended claims.

An object of the present invention is to devise a molecule with sunscreen properties which is covalently attached to an inorganic particle carrier, the carrier being unreactive and biologically inert. Such arrangement prevents the sunscreen molecule's release into the body, therefore effectively solving one of the major drawbacks of sunscreen formulations.

Another object of the present invention is to devise a sunscreen/carrier arrangement that is easily manufactured, preferably in a single step.

Still another object of the present invention is to devise a sunscreen/carrier arrangement which is sufficiently small to result in a transparent, non-gritty formulation, free of rough or sharp surfaces that could damage biopolymers or tissue.

The present invention accomplishes all the objectives mentioned herein above by incorporating the organic sunscreen molecule or dye into spherical silica or organosilica spheres through up to six covalent bonds. The large number of covalent linkages allows the dye to be secured within the arrangement so that leaching will not occur. Furthermore, the dye molecule is not attached to the surface, but is incorporated into the structure of the spheres making it even more difficult to leach out.

### BRIEF DESCRIPTION OF THE SEVERAL VIEWS OF THE DRAWINGS

To the extent that the present application includes any drawing(s), they: (1) are incorporated into and form a part of the specification; (2) illustrate an embodiment of the present invention and; (3) together with the description, serve to explain the principles of the invention. The drawings are only for the purpose of illustrating a preferred embodiment of the invention and are not to be construed as limiting the invention.

FIG. 1 depicts the polymerization of the dye monomer and silica monomers into the composition of the present invention.

### DETAILED DESCRIPTION OF THE DRAWINGS AND INVENTION

The present invention comprises an organic sunscreen molecule or dye, which is incorporated into spherical silica or organosilica spheres through multiple covalent bonds. Up to six covalent bonds allow the dye to be secured within the arrangement. The dye molecule is incorporated into the structure of the spheres, preventing the dye from leaching out.

The sunscreen dye or a mixture of dyes can be formulated to absorb ultraviolet light, including all of the harmful wavelengths (200-400 nanometers; shorter wavelengths are absorbed by the silica in the particles) and even wavelengths of visible light that might prove to be harmful. Only small quantities (<1 mole % or 1 part per 100) of the dye relative to the silica or silsesquioxane co-monomer are required to block the ultraviolet light.

The spherical particles are prepared in one step from the silica or silsesquioxane monomer and the sunscreen dye monomer using a novel anhydrous polymerization. That chemical reaction, as further discussed below, permits spherical nanoparticles of silica or organosilica to be prepared under relatively mild, acidic conditions.

Silica gels prepared from tetraalkoxysilanes and 2-4 equivalents of formic acid, and silsesquioxanes from bridged trialkoxysilanes and 3-6 equivalents of formic acid have previously been reported. [(a)Sharp, Kenneth G. Star alkoxysilane molecules, gels and appreciably tough glasses. Journal of Materials Chemistry (2005), 15(35-36), 3812-3820. (b) Loy, Douglas A.; Russick, Edward M.; Yamanaka, Stacey A.; Baugher, Brigitta M.; Shea, Kenneth J. Direct Formation of Aerogels by Sol-Gel Polymerizations of Alkoxysilanes in Supercritical Carbon Dioxide. Chemistry of Materials (1997), 9(11), 2264-2268.] However, these materials were prepared as monolithic gels in neat (solvent free) or supercritical carbon dioxide solution. In the latter case, silica and phenylene-bridged polysilsesquioxane aerogels were prepared. Those aerogels appeared to comprise spherical particles. However, no attempt was made to isolate the spherical particles, or convert the aerogels into free flowing particles. There is a report of directly preparing silica particles in supercritical carbon dioxide using formic acid, but the particles were polydisperse. [Moner-Girona M.; Roig, A.; Molins, E.; Llibre, J. Sol-Gel Route to Direct Formation of Silica Aerogel Microparticles Using Supercritical Solvents. Journal of Sol-Gel Science and Technology (2003), 26(1/2/3), 645-649.] One of the most important new and useful features of the invention embodied in the present application is the formation of micelles with alkoxysilane monomers, toluene, and formic acid. The present application discloses and claims the miscelles resulting from the homo-polymerization of tetramethoxysilane, tetraethoxysilane, methyltriethoxysilane, methyltrimethoxysilane, 1,1 -bis(triethoxysilyl)methane, 1,1 -bis(trimethoxysilyl)methane, 1,2-bis(triethoxysilyl)ethane, 1,2-bis(trimethoxysilyl)ethane, 1,2-bis(triethoxysilyl)ethene, 1,2-bis(trimethoxysilyl)ethene, 1,6-bis(triethoxysilyl)hexane, 1,6-bis(trimethoxysilyl)hexane, 1,8-bis(triethoxysilyl)octane, 1,8-bis(trimethoxysilyl)octane, 1,10-bis(triethoxysilyl)decane, 1,10-bis(trimethoxysilyl)decane, 1,4-bis(triethoxysilyl)benzene, 1,4-bis(trimethoxysilyl)benzene, 4,4′-bis(triethoxysilyl)biphenyl, 4,4′-bis(trimethoxysilyl)biphenyl, 9,10-bis(triethoxysilyl)anthracene, 9,10-bis(trimethoxysilyl)anthracene, 1,2-bis(4-(triethoxysilyl)phenyl)ethene, 1,2-bis(4-(trimethoxysilyl)phenyl)ethene, 4,4′-bis(4-(triethoxysilyl)styryl)biphenylene, 4,4′-bis(4-(trimethoxysilyl)styryl)biphenylene, 9,10-bis(4-(triethoxysilyl)styryl)anthracene, 10-bis(4-(trimethoxysilyl)styryl)anthracene, N,N′-bis(3-triethoxysilylpropyl)-perylene-3,4:9,10-tetracarboxdiimide, bis(3-trimethoxysilylpropyl)disulfide, bis(3-triethoxysilylpropyl)disulfide, bis(3-trimethoxysilylpropyl)tetrasulfide and bis(3-triethoxysilylpropyl)tetrasulfide into spherical particles.

The chemical reaction disclosed and claimed in the present application does not require addition of water (save a few percent in the formic acid) or surfactants. Specifically, Applicants have found that the formic acid reacts directly with the alkoxide groups to afford the formate ester and a silanol that subsequently reacts with another silanol to afford a siloxane bond. The micelles act as the polar nanoreactors, thus allowing the formic acid, the silanols and the growing particles to react. Because the micelles are formed quickly and are all the same size, the particles form and grow to the same size during the emulsion sol-gel polymerization. Polymerizations are finished in a few hours and the particles can be collected (up to 95% isolated yield) by filtration. Any remaining, non-reacting monomer(s) are nonpolar and reside in the toluene phase.

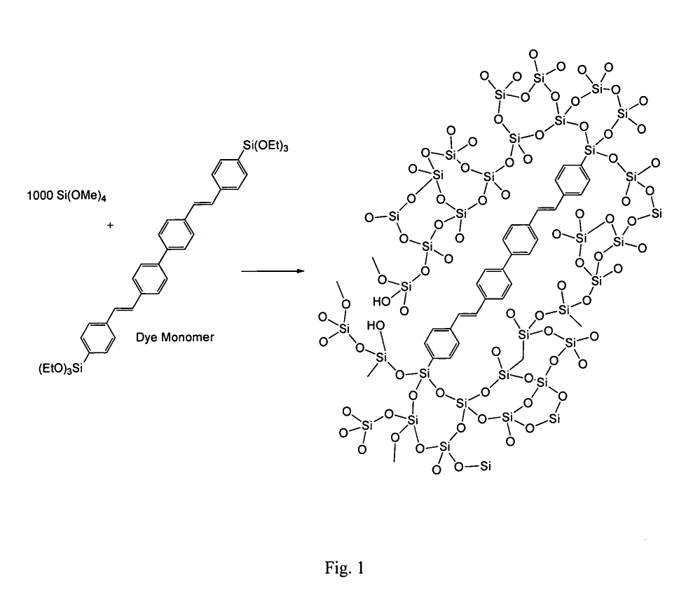

The particles resulting from the chemical reaction illustrated and detailed above are relatively monodisperse. Accordingly, any aggregates can easily be reduced by sonicating for several minutes. The size of the particles is directly related to the concentration of the monomer in the polymerization solution as illustrated in FIG. 1.

The synthetic approach disclosed and claimed in this application differs from prior silica nanoparticle syntheses by the ease with which it can be scaled up. Most nanoparticle preparations use a Stober sphere approach that involves using small concentrations of monomer (<0.01 Moles/Liter) in ethanol with excess ammonia and excess water in a small scale reaction that typically affords 100-200 milligrams of particles. Stober syntheses are susceptible to contaminants or small variations in formulation or environment, making reproducibility difficult. In contrast, the hydrous formic acid/toluene micelle approach disclosed and claimed herein efficiently forms particles with concentrations of monomer (2 Moles/Liter) that would form gels under Stober conditions. In addition, the chemical reaction disclosed and claimed herein is readily scalable. Traditional emulsion polymerization with surfactants to create and stabilize the micelles can also be scaled up, but introduces large quantities of surfactant as a contaminant that is nearly impossible to eliminate.

The method of the present invention can be carried out with any chromophore dye that absorbs in the UV range and can be modified with two or more trialkoxysilyl groups including, but not limited to, dyes with conjugated pi-systems for bridging groups. Examples of dyes Applicants have used include: 1,2-bis(4-(triethoxysilyl)phenyl)ethene, 1,2-bis(4-(2-triethoxysilylvinyl)phenyl)diazene, 1,2-bis(4-(2-triethoxysilylvinyl)phenyl)ethene, 4,4′-bis(4-(triethoxysilyl)vinyl)biphenylene, 4,4′-bis(4-(triethoxysilyl)styryl)biphenylene, 9,10-bis(4-(triethoxysilyl)styryl)anthracene, 9,10-bis(4-(triethoxysilyl)vinylanthracene, and N,N′-bis(3 -triethoxysilylpropyl)-perylene-3,4:9,10-tetracarboxdiimide and the trimethoxysilyl analogs of the above dyes.

The monomers are designed with covalent bonds between the silicon that will form siloxane bonds to the silica or hybrid matrix. Each trialkoxysilyl group can hydrolyze and form up to three siloxane bonds with the matrix. Each monomer has two trailkoxysilyl groups meaning that the dyes can be bonded to the silica or hybrid network by up to six siloxane bonds. The prerequisite of breaking six siloxane bonds before extracting the dye from the silica matrix virtually eliminates leaching of the sunscreen dye from the particles.

Silica nanoparticles prepared under alkaline conditions are generally non-porous. Accordingly, the particles' overall surface area (m²/g) is due to the exterior surfaces of the individual spherical particles. The new and useful family of silica nanoparticles and the method of preparing the same disclosed and claimed herein results in particles between 200 nanometers and 5 microns. Further, the resulting particles appear to be highly porous rather than solid silica. The nanoparticles' usefulness as a sunscreen, therefore, can be greatly enhanced by using the pores within the particles to deliver any desired chemicals, including but not limited to moisturizing agents.

Porosity in particles can be identified easily by anomalously high measured surface areas compared to those calculated from the non-porous particle diameters. Assuming close packing of spherical particles, surface area increases as a function of particle radius according the following equation:

Surface area in 1 gram˜4πr² (4/3 πr³·density)=3/(r·density) in square meters (m²).

For a gram of silica (d=2.65 g/mL=2,650,000 g/m³) particles 200 nanometers in diameter (r=0.0000001 meters), the surface area would be 11.3 m² and all of the surface area would be due to the spherical geometry of the particles and how well they pack into a space.

The particles prepared by the method disclosed and claimed in this application were characterized by nitrogen sorption porosimetry to have surface areas over 247 m²/g (calculated surface area=3 m 2/g) and as high as 575 m²/g (calculated surface area=4 m²/g). The surface area analyses revealed type I isotherms indicative of microporous (pores<20 nm diameter) materials and BJH analyses of the data revealed mean pore size diameters between 10-15 nanometers.

The pores in the silica nanoparticles of this invention appear to be several orders of magnitude smaller than the diameters of the particles themselves. That means that the particles are likely to be sponge-like structures. The large number of pores and the fact that they are large enough to accommodate any solvents, synthetic pharmaceuticals, surfactants, and even many proteins means that the porosity in the particles could be used to hold a reservoir of chemicals whose release would be desirable. For example, chemical compositions with moisturizing, anti-itch, analgesic, anti-bacterial, anti-wrinkle, emollient, nutrient or other desirable properties can be stored in the porous particles to extend and diversify the efficacy of a sunscreen formulation.

### ILLUSTRATIVE EXPERIMENTAL PROTOCOLS

### 1. General Formic Acid Procedure at Lower Concentration: 0.1 M Tetramethoxysilane and 8 M Formic Acid

Spheres were prepared by mixing together two separate solutions, A and B. Solution A contained tetramethoxysilane (TMOS, 0.1522 g, 0.001 moles, 0.1 M in tetramethoxysilane) that was diluted to 5 mL with anhydrous toluene/tetrahydrofuran (75:25) mixture. Solution B contained the acidic catalyst formic acid (3.68 g, 0.08 moles, 8.0 M in formic acid) that was diluted to 5 mL with anhydrous toluene/tetrahydrofuran (75:25) mixture. Solution B was added quickly to solution A and mixed for approximately thirty seconds bringing the total volume to 10 mL.

### 2. General Formic Acid Procedure at Higher Concentrations 2.0 M Tetramethoxysilane and 8 M Formic Acid

Spheres were prepared by mixing together two separate solutions, A and B. Solution A contained tetramethoxysilane (TMOS, 3.04 g, 0.02 moles, 2.0 M in tetramethoxysilane) that was diluted to 5 mL with anhydrous toluene. Solution B contained the acidic catalyst formic acid (3.68 g, 0.08 moles, 8.0 M in formic acid) that was diluted to 5 mL with anhydrous toluene. Solution B was added quickly to solution A and mixed for approximately thirty seconds, bringing the total volume to 10 mL.

3. Formic Acid Procedure with Base Sensitive Silsesquioxane Monomer: 0.01 M Tetrasulfide 8 M Formic Acid

Spheres were prepared by mixing together two separate solutions, A and B. Solution A contained tetrasulfide bridged monomer (0.0538 g, 0.0001 moles, 0.01 M in monomer) that was diluted to 5 mL with anhydrous toluene. Solution B contained the acidic catalyst formic acid (3.68 g, 0.08 moles, 8.0 M in formic acid) that was diluted to 5 mL with anhydrous toluene. Solution B was added quickly to solution A and mixed for approximately thirty seconds bringing the total volume to 10 mL.

4. 1 M TMOS, 8 M Formic Acid, with Fluorescent Dye

Spheres were prepared by mixing together two separate solutions, A and B. Solution A contained tetramethoxysilane (TMOS, 22.5 g, 0.15 moles, 2.0 M in tetramethoxysilane) and 4,4′-bis(4-(triethoxysilyl)styryl)biphenylene (0.1 gram, 0.15 mmoles, 0.1 mol%) that was diluted to 75 mL with anhydrous toluene. Solution B contained the acidic catalyst formic acid (27.6 g, 0.6 moles, 8.0 M in formic acid) that was diluted to 75 mL with anhydrous toluene. Solution B was added quickly to solution A and mixed for approximately thirty seconds bringing the total volume to 150 mL. Particles formed within a few hours. The reaction was filtered to afford the product as a UV-fluorescent (blue), white powder (9 g, 100% yield). Particles were determined by SEM to be 2.5 micrometers in diameter.

5. 0.01 M TEOS, Stober Conditions with Fluorescent Dye

Spheres were prepared by mixing together two separate solutions, A and B. Solution A contained tetraethoxysilane (TEOS, 0.355 g, 1.7 mmoles) and 4,4′-bis(4-(triethoxysilyl)styryl)biphenylene (0.0012 gram, 1.7 μmoles, 0.1 mol%) that was diluted to 5 mL with anhydrous ethanol. Solution B contained ammonia in ethanol (2 M g, 3.75 mL) and water (1.01 mL). Solution B was added quickly to solution A and mixed for approximately thirty seconds, bringing the total volume to 10 mL. Particles formed within a few hours. The reaction was filtered to afford the product as a UV-fluorescent (blue), white powder (<0.05 g, <50% yield). Particles were 400 nm in diameter.

What I claim is:

1. A chemical composition with sunscreen properties which can be manufactured in a single step, the composition comprising:
a. an organic molecule;
b. an inorganic particle carrier capable of being covalently attached to the organic molecule, the carrier being non-reactive and biologically inert, so that when the composition is applied to human skin, the organic molecule cannot be released into the user's body.

2. The chemical composition according to claim 1, wherein the organic molecule-carrier arrangement is sufficiently small to result in a transparent, non-gritty formulation, free of rough or sharp surfaces that could damage biopolymers or tissue.

3. The chemical composition according to claim 1, wherein the organic molecule comprises a dye, and the inorganic carrier is selected from the group including spherical silica and organosilica spheres, and where the organic molecule is covalently attached to the structure of the inorganic carrier through up to six covalent bonds, preventing leaching of the dye.

4. The chemical composition according to claim 1, wherein the organic molecule comprises a sunscreen dye or a mixture of dyes capable of absorbing ultraviolet light, including all of the frequencies harmful to humans, including visible light; and wherein the inorganic carrier comprises spheres capable or absorbing shorter frequency ultraviolet light.

5. The chemical composition according to claim 1, wherein the carrier is highly porous, the pores being several orders of magnitude smaller than the diameter of the carrier itself resulting in a sponge-like carrier, the pores being large enough to accommodate and carry molecules selected from the group consisting of solvents, synthetic pharmaceuticals, surfactants, and proteins, thus allowing the carrier to be used as a reservoir of chemical compositions capable of being released upon application by a user, the chemical compositions having moisturizing, anti-itch, analgesic, anti-bacterial, anti-wrinkle, emollient, nutrient or other desirable properties.

6. The chemical composition according to claim 1, wherein the organic molecule comprises a chromophore dye that absorbs in the UV light range, the chromophore being capable of being modified with two or more trialkoxysilane groups, the chromophore comprising dyes with conjugated pi-systems for bridging groups such as 1,2-bis(4-(triethoxysilyl)phenyl)ethene, 1,2-bis(4-(2-triethoxysilylvinyl)phenyl)diazene, 1,2-bis(4-(2-triethoxysilylvinyl)phenyl)ethene 4,4′-bis(4-(triethoxysilyl)vinyl)biphenylene, 4,4′-bis(4-(triethoxysilyl)styryl)biphenylene, 9,10-bis(4-(triethoxysilyl)styryl)anthracene, 9,10-bis(4-(triethoxysilyl)vinylanthracene and N,N′-bis(3-triethoxysilylpropyl)-perylene-3,4:9,10-tetracarboxdiiniide, other aromatic analogs, and those with trimethoxysilylgroups instead of triethoxysilyl groups.

7. A single-step method of preparing an inorganic carrier to be used as a component of a chemical composition with sunscreen properties via the homo-polymerization of a silica containing compound into spherical particles, the method comprising the steps of:
a. preparing a solution A, solution A comprising a diluted alkoxysilane composition;
b. diluting the composition to a predetermined volume with an anhydrous solvent selected from the group consisting of toluene and toluene/tetrahydrofuran (75:25 mixture);
c. preparing a solution B, solution B comprising an acidic catalyst formic acid diluted to a predetermined volume with an anhydrous solvent selected from the group consisting of toluene and toluene/tetrahydrofuran (75:25 mixture);
d. mixing solution A and solution B by adding solution B quickly to solution A and mixing for approximately thirty seconds bringing the total volume to double the predetermined volumes.
